(19) United States
(12) Patent Application Publication
(10) Pub. No.: US 2010/0137889 A1
(43) Pub. Date: Jun. 3, 2010

(54) Surgical Instrument and Method for Attaching Soft Tissue to a Bone

(75) Inventors: Israel Oren, Kibbutz Gaaton - Doar-Na Oshrat (IL); Dan Moor, Kibbutz Gaaton - Doar-Na Oshrat (IL); Lee Ranon, Nahariya (IL); Eran Zakai, Moshav Yodfat - Doar-Na Misgav (IL); Laurent Lafosse, Annecy-Le-Vieux (FR)

Correspondence Address: MARTIN D. MOYNIHAN d/b/a PRTSI, INC., P.O. BOX 16446, ARLINGTON, VA, 22215, US

(73) Assignees: T.A.G. Medical Products a Limited Partnership, Kibbutz Gaaton--Doar-Na Oshrat (IL); Ran Oren, Kibbutz Gaaton-Doar-Na Oshrat (IL); Dan Moor, Kibbutz Gaaton- Doar-Na-Oshrat (IL)

(21) Appl. No.: 12/224,943

(22) PCT Filed: Mar. 14, 2007
(86) PCT No.: PCT/IL2007/000335

§ 371 Date: Mar. 16, 2009

Related U.S. Application Data
(60) Provisional application No. 60/783,035, filed on Mar. 17, 2006.

(51) Int. Cl. A61B 17/04

(52) U.S. Cl. 606/145; 606/148

(57) ABSTRACT

A surgical instrument for use in attaching soft tissue to a bone in a surgical site, includes an elongated shaft carrying, at its distal end, a clamping device having clamping jaws and a piercing device for piercing soft tissue, and further carrying, at its proximal end, manipulatable members for manipulating the clamping device to clamp soft tissue between the clamping jaws, and for manipulating the piercing device for piercing the soft tissue when clamped between the clamping jaws, and for drawing a suture through the pierced soft tissue. The surgical instrument further includes, at its distal end, a socket dimensioned to receive an anchor to be implanted in a bore in the bone, which anchor also has secured thereto a suture to be passed through the pierced soft tissue and to be tied to the bone. The invention is particularly described with respect to a method wherein the tissue to be attached is labrum tissue detached from the glenoid bone in a shoulder joint.

### FIELD AND BACKGROUND OF THE INVENTION

The present invention relates to a surgical instrument, and also to a method, for attaching soft tissue to a bone. The invention is particularly useful, and is therefore described below, with respect to the repair of labral tears in shoulder joints, in which the labrum of the shoulder joint is to be surgically reattached to the glenoid bone, but it will be appreciated that the invention could advantageously be used in other applications as well.

Minimally-invasive tissue repair procedures, such as arthroscopic and endoscopic procedures, are generally preferred whenever possible in order to minimize trauma and damage to surrounding tissue layers, thus shortening the time needed for recovery. This is particularly true with respect to the repair of labral tears in shoulder joints.

The range of movements the human shoulder can make far exceeds any other joint in the body. The shoulder joint is a ball and socket joint, similar to the hip; however, the socket of the shoulder joint is extremely shallow, and thus inherently unstable. Muscles and tendons serve to keep the bones in approximation. In order to compensate for the shallow socket, the shoulder joint has a cuff of fibrous cartilage called a labrum that forms a cup for the head of the arm bone (humerus) to move within. This cuff of cartilage makes the shoulder joint much more stable, yet allows for a very wide range of movements. When the labrum of the shoulder joint is damaged, the stability of the shoulder joint is compromised.

Shoulder dislocations often tear the labrum, especially in younger patients. The labrum tear, called a Bankart lesion, in most cases involves the part of the labrum called the inferior glenohumeral ligament. The lesion is seen in over 85% of cases after a traumatic anterior dislocation. The inferior gleno-humeral ligament, which is attached medially to the lower half of the anterior glenoid labrum, is the most important of the ligaments that stabilize the shoulder. At the time of the original injury, the humeral head, when it is forced out anteriorly and inferiorly, first stretches the anterior capsule and the inferior glenohumeral ligament. Then, as a result of traction, the fibrous labrum is pulled off from the inferior half of the anterior rim of the glenoid. The damage suffered, if not treated, may cause recurring dislocations.

With past mid-age sedentary patients, conventional treatment may be recommended; but with younger and physically active patients, surgical intervention is usually necessary to restore the stability of the shoulder joint to full function. The aim of the operation is to re-attach the separated part of the labrum to the glenoid at its normal anatomical position.

The procedure for repairing a labral tear arthroscopically involves the following steps:

(1) mapping the joint and opening portals for visualization, irrigation and for the instruments through small cuts in the skin;

(2) drilling bores in the glenoid rim;

(3) inserting bone anchors with sutures attached to each anchor into the bores;

(4) grasping the torn labrum and moving it back into its original position on the glenoid; and

(5) bringing suture strands coming from the anchor through the tissue to the outside using a suture retriever and tying the strands to attach and tighten the labrum to the glenoid rim.

Several prior art devices are available to perform each step of the process separately: anchor inserters, graspers, suture retrievers, etc. Examples of such prior art devices are described in U.S. Pat. Nos. 6,511,487 and 5,499,991 and in US Published Patent Applications 2005/0043748 and 2002/0065526.

### OBJECTS AND BRIEF SUMMARY OF THE PRESENT INVENTION

An object of the present invention is to provide a single surgical instrument capable of performing all the necessary steps in attaching soft tissue to a bone in a surgical site, particularly steps (3)-(5) in the above-described procedure for repairing a labral tear. Another object of the invention is to provide a method of attaching soft tissue to a bone, which method is particularly useful in the above-described procedure for repairing labral tears in shoulder joints.

According to one aspect of the present invention, there is provided a surgical instrument for use in attaching soft tissue to a bone in a surgical site, comprising: an elongated shaft having a distal end carrying a clamping device including clamping jaws, and a piercing device for piercing soft tissue; the elongated shaft having a proximal end carrying manipulatable members for manipulating the clamping device to clamp soft tissue between the clamping jaws, and for manipulating the piercing device for piercing the soft tissue when clamped between the clamping jaws, and for drawing a suture through the pierced soft tissue; characterized in that the surgical instrument further includes, at the distal end of the elongated shaft, a socket dimensioned to receive an anchor to be implanted in a bore in the bone, which anchor also has secured thereto a suture to be passed through the pierced soft tissue and to be tied to the bone.

According to a more particular aspect of the present invention, there is provided a surgical instrument for attaching soft tissue to a bone at a surgical site, comprising: an elongated shaft having a proximal end to be grasped by a user of the surgical instrument, and a distal end to be introduced into the surgical site; a socket carried at the distal end of the elongated shaft and dimensioned for removably receiving an anchor to be secured to the bone at the surgical site, which anchor has a suture secured thereto; a clamping device including a pair of clamping jaws carried by the distal end of the elongated shaft and movable to open and closed positions with respect to each other; a piercing device including a crochet head carried by the elongated shaft and movable in the distal and proximal directions with respect thereto; the crochet head having a pointed tip for piercing a hole through soft tissue clamped between the jaws when in the closed position and when the crochet head is moved in the distal direction, and a hook formation for catching the suture and for drawing it through the pierced hole when the crochet head is moved in the proximal direction; and manipulatable members carried by the proximal end of the elongated shaft for moving the jaws to their open and closed positions, and for moving the crochet head in the distal and proximal directions.

According to yet another aspect of the present invention, there is provided a method of surgically attaching soft tissue to a bone at a surgical site, comprising: drilling a bore in the bone; inserting into the surgical site a surgical instrument carrying at its distal end an anchor secured to an intermediate portion of a suture, a tissue-clamping device including a pair of clamping jaws, and a crochet head having a pointed tip; implanting the anchor in the bore of the bone; manipulating the clamping device to clamp a selected portion of the tissue between the clamping jaws; manipulating the crochet head to pierce the selected portion of the clamped tissue, and to draw one end of the suture therethrough; removing the surgical instrument from the surgical site; knotting the two sends of the suture; and sliding the knot to the bone to tighten the tissue to the bone.

The surgical instrument and method are described below with respect to a preferred embodiment for the repair of labral tears wherein the tissue to be attached is a portion of the labrum separated detached from the glenoid bone in a shoulder joint.

Further features and advantages of the invention will be apparent from the description below.

### BRIEF DESCRIPTION OF THE DRAWINGS

The invention is herein described, by way of example only, with reference to the accompanying drawings, wherein:

FIG. 1 schematically illustrates the anatomy of a shoulder joint;

FIG. 2 is a side elevational view illustrating one form of surgical instrument constructed in accordance with the present invention;

FIG. 3 is an enlarged fragmentary view of the distal end of the surgical instrument of FIG. 2;

FIG. 4 illustrates the distal end of the surgical instrument of FIG. 3 in a particular stage of its use, wherein labral tissue, clamped between the jaws of the instrument, is about to be pieced by the piercing device of the surgical instrument;

FIG. 5 schematically illustrates one technique of suturing using the surgical instrument of the FIGS. 2-4; and

FIG. 6 illustrates an alternative method of suturing using the suturing instrument of FIGS. 2-4.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### The Anatomy of a Shoulder Joint

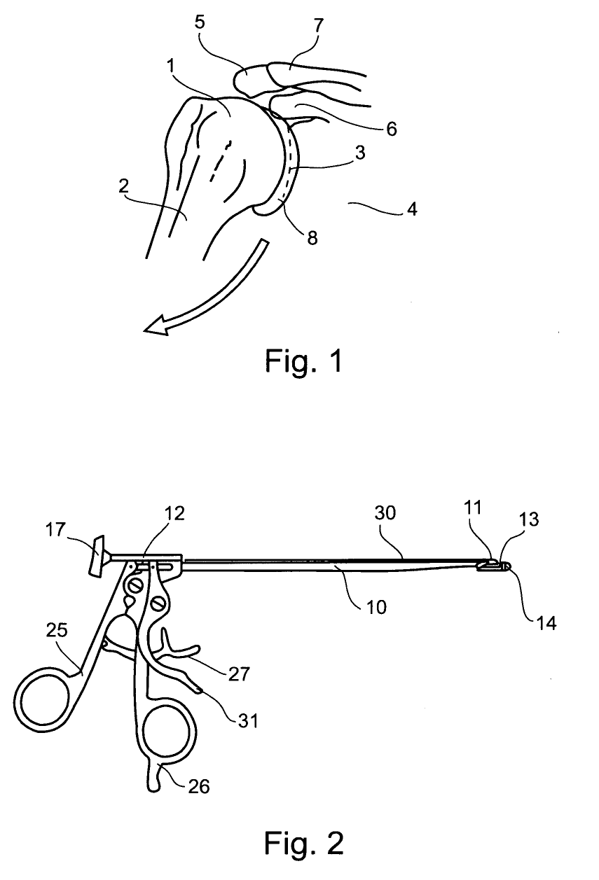

FIG. 1 illustrates the anatomy of a shoulder joint. The head 1 of the upper arm bone or humerus 2, forms a ball-and-socket joint with the shallow glenoid cavity 3. The glenoid is the lateral part of the shoulder blade scapula 4. Two hook-like projections of the scapula overhanging the glenoid are the acromion 5 and the coracoid process 6. A group of muscles, collectively know as the Rotator Cuff, originate on the scapula and insert on the humerus. These serve to stabilize the joint by keeping the humeral head in contact with the glenoid cavity. The clavicle 7 connects the acromion to the breastbone sternum. The glenoid labrum 8, which is a flexible fibrous ligament, surrounds the glenoid rim enlarging its area of contact with the humerus. When dislocations in the direction shown by the arrow occur, the anterior-inferior part of the labrum is torn away from the glenoid, causing instability of the joint.

Portals to be used in the repair procedure are placed relative to the bony structures marked out on the skin. The portals must provide safe access to the surgical site at an angle suitable for work.

Of the various steps involved in the procedure for repairing a labral tear arthroscopically as briefly described above, the surgical instrument illustrated in FIGS. 2-4 of the drawings is particularly useful for performing steps (3)-(5).

### DESCRIPTION OF A PREFERRED EMBODIMENT

### The Overall Construction

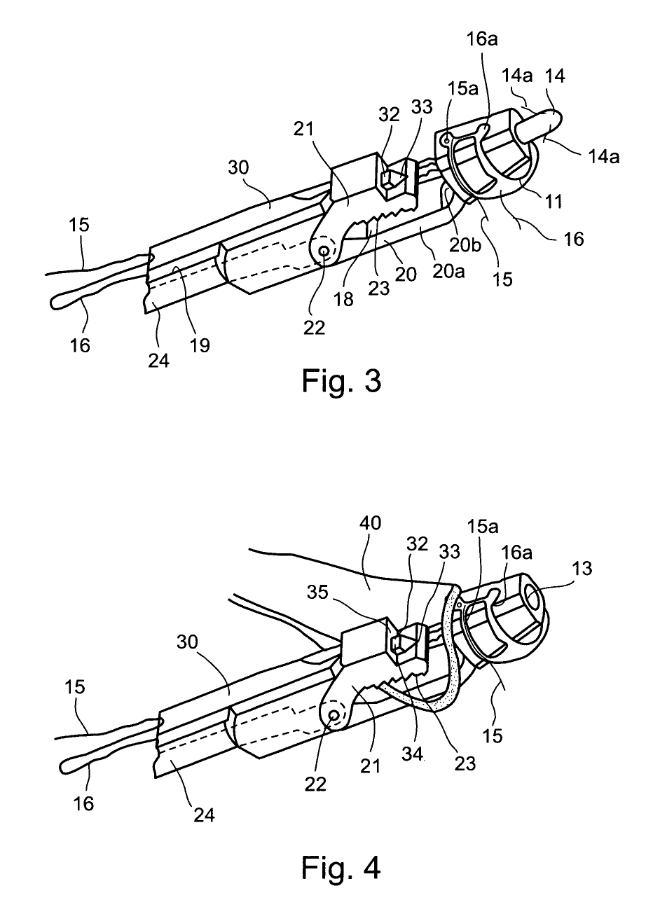

FIG. 2 illustrates one form of surgical instrument constructed in accordance with the present invention; whereas FIGS. 3 and 4 illustrate the distal end of the instrument.

As shown in FIG. 2, the surgical instrument includes an elongated shaft 10 having a distal end 11 for insertion into the surgical site, and a proximal end 12 to be located outside of the surgical site for manipulating the surgical instrument and the various parts thereof, as will be described more particularly below.

Elongated shaft 10 is made of a rigid material, e.g., metal, of a suitable cross-section. Its distal tip is formed with a socket 13 (FIG. 4) dimensioned to receive an anchor 14 (FIG. 3), to be force-fitted into a bore formed in the bone (glenoid) to which soft tissue (fibril tissue) is to be attached. As shown in FIG. 3, anchor 14 includes barbs 14a, 14b, for securely anchoring it in place. In addition, the anchor includes two sutures 15, 16, to be used for attaching the labral tissue to the bone receiving the anchor.

The distal tip 11 of elongated shaft 10 is further formed with two slots 15a, 16a, for receiving the free ends of the two sutures, 15, 16, respectively, fixed to the anchor 14.

The proximal end 12 of elongated shaft 10 is further provided with a finger-piece 17 to be used, as will be described below, for force-fitting the anchor 14 into a bore formed in the bone to which the soft tissue is to be attached. It will be seen in FIG. 2 that the longitudinal axis of the finger-piece 17 and of the proximal portion 12 of elongated shaft 10 is substantially parallel to the longitudinal axis of the socket 13 at the distal end 11 of the elongated shaft. This facilitates the force-fitting of the anchor 14 into the bore in the bone by an axial pressure applied, e.g., via the user's thumb, to the finger-piece 17.

Elongated shaft 10 is further formed, at its distal end, with a recess 18 immediately proximally to the two slots 15a, 16a receiving the sutures 15, 16. As will be described below, recess 18 in elongated shaft 10 defines one jaw of a clamping device carried at the distal end of the elongated shaft for engaging and clamping a portion of the soft tissue (e.g., labral tissue) to be attached to the bone in the surgical site.

Elongated shaft 10 is further formed with a channel 19 on its upper face extending from its proximal end 12 to its distal end 11. As will be described below, channel 19 accommodates a piercing device, in the form of a crochet head, used for piercing the portion of the soft tissue clamped by the clamping device, and for drawing therethrough one of the sutures 15, 16 from the anchor 14.

As shown particularly in FIG. 3, recess 18 at the distal end 11 of elongated shaft 10 is shaped to define a fixed jaw 20 having an axially-extending portion 20a and a transversely-extending portion 20b at the distal end of the recess. The distal end of elongated shaft 10 further includes a pivotal jaw 21 pivotally mounted at 22, at the proximal end of recess 18, to a closed or clamping position with respect to fixed jaw 20, or to an open or unclamping position with respect to the fixed jaw. The under-face of pivotal jaw 21 is grooved or serrated, as shown at 23, to firmly clamp the soft tissue therebetween when the clamping device is in its closed, clamping condition.

Pivotal jaw 21 is coupled to one end of a link 24 extending the along the length of the elongated shaft 10. The opposite end of link 24 is coupled to a handle 25 pivotally mounted at the proximal end of the elongated shaft. Handle 25 cooperates with a second handle 26 fixed to the elongated shaft at its proximal end, such that moving handle 25 towards handle 26 pivots jaw 21 towards the fixed jaw 20 to clamp any soft tissue therebetween, and moving handle 25 away from handle 26 pivots jaw 21 to its open position to release the tissue.

As further shown in FIG. 2, the proximal end of elongated shaft 10 further includes a ratchet arm 27 pivotally carried by handle 25 and engageable with handle 26, to releasably retain the two handles in any moved position, and thereby to releasably retain any soft tissue clamped between the two jaws 20, 21 irrespective of the thickness of the tissue.

The piercing device 30 included in the illustrated surgical instrument is in the form of an elongated member (FIGS. 3, 4) which is longitudinally movable within channel 19 formed in the upper face of elongated shaft 10 in the distal and proximal directions. For this purpose, elongated member of piercing device 30 is coupled, at the proximal end of the elongated shaft, to a pivotal arm 31 (FIG. 2), such that moving arm 31 towards handle 25, pushes the piercing device 30 in the distal direction, and moving arm 31 away from handle 25 pulls the piercing device 30 in the proximal direction.

Piercing device 30 is used for piercing the tissue clamped between jaws 20 and 21 of the clamping device at the distal end of the surgical instrument, and for drawing a suture through the hole so formed. Thus, as shown in FIGS. 3 and 4, the distal end of piercing device 30 carries a crochet head 32 formed with a pointed tip 33 for piercing the tissue clamped between the jaws 20, 21 during the movement of the piercing device in the distal direction. Crochet head 32 is further formed with a hook formation 34 for engaging one of the sutures 15, 16, and for drawing same back through the so-formed pierced opening during the movement of the piercing device in the proximal direction. The movements of the piercing device 30 are guided by a channel 35 formed in the upper surface of pivotal jaw 21.

### Examples of the Manner of Use

As indicated earlier, the surgical instrument illustrated in FIGS. 2-4 may be used in performing steps 3-5 described above for repairing a labral tear arthroscopically. Steps 1 and 2 are performed in a conventional manner. The appropriate number of bores would be drilled in the glenoidal rim, each bore receiving one of the anchors 14 (FIG. 3) to be used in the surgical procedure.

For each such bore, the surgical instrument is loaded with the appropriate anchor 14 inserted into socket 13 at the distal end of elongated shaft 10, and its sutures 15, 16 are received within their respective slots 15a, 16a at the distal end of the elongated shaft. In the example described herein, the soft tissue to be attached is pierced at two points for each anchor to receive the two sutures 15, 16, as shown in FIG. 6. It will be appreciated, however, that in some applications it may be sufficient to pierce the tissue at only one point, in which case only one suture, e.g., 15, would be drawn through the pierced opening in the tissue to be attached.

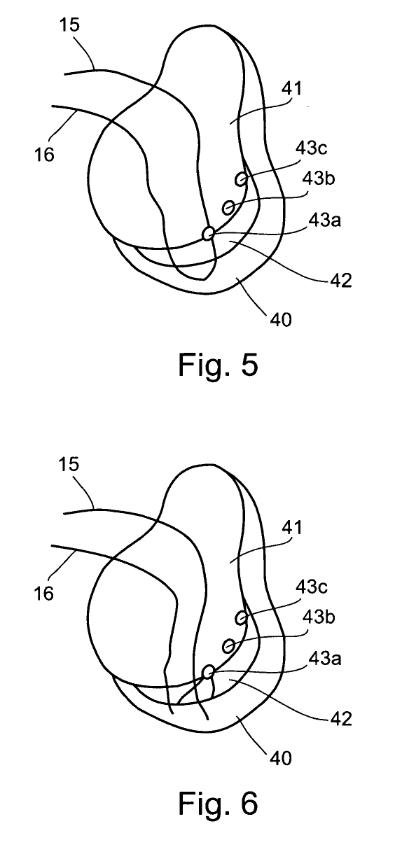

FIG. 5 schematically illustrates the labral tissue 40 separated from the glenoid 41, because of a labral tear, producing a gap 42, which is to be arthroscopically repaired by inserting anchors 14 into bores formed in the glenoid 41, passing sutures through the fibril tissue and tying same to the glenoid. FIG. 5 illustrates three bores 43a-43c for receiving three anchors 14, but it will be appreciated that any appropriate number of anchors can be used according to the particular condition to be repaired.

After the illustrated surgical instrument is loaded with the first anchor 14, the anchor is inserted into the lower-drilled bore 43a, as shown in FIG. 5, and is fixed therein by applying pressure to finger-piece 17 at the proximal end of the instrument.

Jaws 20, 21 of the clamping device are now opened via handles 25, 26, and the instrument is positioned within the shoulder joint so that the fixed jaw 20 lies under the inferior separated part of the labrum 40, while the movable jaw 21 is above the surface of the labrum.

The jaws are then closed, whereupon the labral tissue 40 is grasped between the jaws and is folded against the distal shoulder 20b defined by the recess 18 formed in the fixed jaw member 20 (FIG. 4). The tissue is then manipulated to lie over its original anatomic position.

With the device held in this position, the surgeon advances piercing device 30 in the distal direction by actuating lever 31 to cause the pointed tip 33 of the crochet head 32 to pierce the tissue. The piercing device then executes the return movement, in the proximal direction, whereupon hook formation 34 of the crochet head 32 engages suture strand 15 in slot 15a and pulls it through the tissue. Opening the jaws 20, 21 now allows the surgeon to disengage the device from the labrum and bring it outside of the body for knotting suture strands 15, 16 with a sliding knot, which is then manipulated to tighten the labrum to the glenoid.

As shown in FIG. 5, suture strand 15 comes directly from the anchor 14, and passes through the tissue 40. Thus, when the sliding knot tightens the labrum to the glenoid, the gap 42 formed between the glenoid 41 and the separated labrum 40 is closed.

In the alternative method of suturing shown in FIG. 6, both suture strands 15, 16 are loaded into their respective slots 15a and 16a respectively. After the first suture strand 15 is brought through the tissue 40 as described above, the jaws are opened. The surgeon now grasps the labrum again at a suitable distance from the exit point of the first suture strand 15 and operates the piercing device 30 to pierce the tissue with a second hole, and to bring the second strand 16 from the second slot 16a through that hole in the tissue. The knot tied between these sutures overlies the labrum as both strands now come through the labrum.

The above procedure is repeated for each anchor to be inserted. It will be seen that the surgical instrument need be removed from the surgical site only when inserting another anchor.

While the invention has been described to one preferred embodiment, it will be appreciated that this is set forth merely for purposes of example, and that many other variations, modifications and applications of the invention may be made.

What is claimed is:

1. A surgical instrument for use in attaching soft tissue to a bone in a surgical site, comprising:
an elongated shaft having a distal end carrying a clamping device including clamping jaws, and a piercing device for piercing soft tissue;
said elongated shaft having a proximal end carrying manipulatable members for manipulating said clamping device to clamp soft tissue between the clamping jaws, and for manipulating said piercing device for piercing the soft tissue when clamped between said clamping jaws, and for drawing a suture through the pierced soft tissue;
characterized in that said surgical instrument further includes, at the distal end of said elongated shaft, a socket dimensioned to receive an anchor to be implanted in a bore in the bone, which anchor also has secured thereto a suture to be passed through the pierced soft tissue and to be tied to the bone.

2. The surgical instrument according to claim 1, wherein said piercing device is a crochet head having a pointed distal tip for piercing tissue clamped between the jaws of said clamping device during the movement of the crochet head in the distal direction.

3. The surgical instrument according to claim 2, wherein said crochet head has a hook formation spaced from its pointed distal tip for receiving the suture, and for drawing same through the pierced tissue during the movement of the crochet head in the proximal direction.

4. The surgical instrument according to claim 1, wherein said distal end of the elongated shaft is formed with a slot extending across the path of movement of the piercing device when moving in the proximal direction, for receiving the suture to be drawn through the tissue after pierced by said piercing device.

5. The surgical instrument according to claim 4, wherein said distal end of the elongated shaft is formed with a second slot for receiving a second suture secured to the anchor, to be drawn through a second hole formed by said piercing device.

6. The surgical instrument according to claim 1, wherein said clamping jaws include a fixed jaw fixed to said elongated shaft, and a pivotal jaw pivotal towards and away from said fixed jaw to closed and open positions with respect thereto.

7. The surgical instrument according to claim 6, wherein said piercing device moves through a channel formed in said pivotal jaw.

8. The surgical instrument according to claim 1, wherein the longitudinal axis of said socket at said distal end of the elongated shaft is parallel to the longitudinal axis of said elongated shaft; and wherein the proximal end of the elongated shaft includes a finger-piece having an axis also parallel to the longitudinal axis of said elongated shaft for pressure-forcing the anchor in said socket into a bore formed in the bone.

9. The surgical instrument according to claim 1, wherein said manipulatable members carried at the proximal end of said elongated shaft include: a first handle fixed to the proximal end of said elongated shaft, and a second handle pivotal to the proximal end of said elongated shaft and coupled to one of said clamping jaws for moving same to closed and open position with respect to said fixed jaw upon pivoting said second handle with respect to said first handle.

10. The surgical instrument according to claim 9, wherein said manipulatable members carried at the proximal end of said elongated shaft further includes a lever arm pivotal to said first handle and coupled to said piercing device for moving same in the distal and proximal directions upon pivoting said lever arm with respect to said first handle.

11. The surgical instrument according to claim 9, wherein said manipulatable members carried by said proximal end of the elongated shaft further includes a ratchet device for releasable retaining said first and second handles in a selected pivoted position with respect to each other.

12. The surgical instrument according to claim 1, wherein said elongated shaft, clamping device and piercing device are dimensioned for repairing a labrum tear in a shoulder joint.

13. A surgical instrument for attaching soft tissue to a bone at a surgical site, comprising:
an elongated shaft having a proximal end to be grasped by a user of the surgical instrument, and a distal end to be introduced into the surgical site;
a socket carried at said distal end of the elongated shaft and dimensioned for removably receiving an anchor to be secured to the bone at the surgical site, which anchor has a suture secured thereto;
a clamping device including a pair of clamping jaws carried by said distal end of the elongated shaft and movable to open and closed positions with respect to each other;
a piercing device including a crochet head carried by said elongated shaft and movable in the distal and proximal directions with respect thereto; said crochet head having a pointed tip for piercing a hole through soft tissue clamped between said jaws when in said closed position and when said crochet head is moved in the distal direction, and a hook formation for catching the suture and for drawing it through the pierced hole when said crochet head is moved in the proximal direction;
and manipulatable members carried by said proximal end of the elongated shaft for moving said jaws to their open and closed positions, and for moving said crochet head in said distal and proximal directions.

14. The surgical instrument according to claim 13, wherein said distal end of the elongated shaft is formed with a slot extending across the path of movement of the piercing device in the proximal direction of that device, for receiving the suture to be drawn through the tissue after pierced by said piercing device.

15. The surgical instrument according to claim 13, wherein said clamping jaws include a fixed jaw fixed to said elongated shaft, and a pivotal jaw pivotal towards and away from said fixed jaw to closed and open positions with respect thereto.

16. The surgical instrument according to claim 15, wherein said piercing device moves through a channel formed in said pivotal jaw.

17. The surgical instrument according to claim 13, wherein the longitudinal axis of said socket at said distal end of the elongated shaft is parallel to the longitudinal axis of said elongated shaft; and wherein the proximal end of the elongated shaft includes a finger-piece having an axis also parallel to the longitudinal axis of said elongated shaft for pressure-forcing the anchor in said socket into a bore formed in the bone.

18. A method of surgically attaching soft tissue to a bone at a surgical site, comprising:
drilling a bore in said bone;
inserting into said surgical site a surgical instrument carrying at its distal end an anchor secured to an intermediate portion of a suture, a tissue-clamping device including a pair of clamping jaws, and a crochet head having a pointed tip;
implanting said anchor in said bore of the bone;
manipulating said clamping device to clamp a selected portion of said tissue between said clamping jaws;
manipulating said crochet head to pierce said selected portion of the clamped tissue, and to draw one end of the suture therethrough;
removing the surgical instrument from the surgical site;
knotting the two sends of the suture;
and sliding the knot to the bone to tighten the tissue to the bone.

19. The method according to claim 18, wherein after said crochet head has been manipulated to pierce said selected portion of the clamped tissue, and to draw one end of the suture therethrough, said crochet head is manipulated to pierce another selected portion of the clamped tissue, and to draw the other end of the suture therethrough.

20. The method according to claim 18, wherein the tissue to be attached is labrum tissue detached from the glenoid bone in a shoulder joint.
